(19) United States
(12) Patent Application Publication
(10) Pub. No.: US 2006/0157584 A1
(43) Pub. Date: Jul. 20, 2006

(54) Liquid-like body-jetting adapter and liquid-like body feeder/container

(76) Inventors: Koji Nomiyama, Tokyo (JP); Hiroshi Komatsuzaki, Tokyo (JP); Takanobu Seki, Tokyo (JP); Junya Takamizawa, Tochigi (JP)

Correspondence Address: STEPTOE & JOHNSON LLP, 1330 CONNECTICUT AVENUE, N.W., WASHINGTON, DC, 20036, US

(21) Appl. No.: 10/545,898

(22) PCT Filed: Feb. 23, 2004
(86) PCT No.: PCT/JP04/02061

§ 371 Date: Aug. 17, 2005

(30) Foreign Application Priority Data
Feb. 25, 2003 (JP) 2003-047933
Apr. 16, 2003 (JP) 2003-111613
Apr. 16, 2003 (JP) 2003-111612
Jun. 25, 2003 (JP) 2003-180903

(51) Int. Cl. A62C 13/62; B05B 7/30; A61M 11/02; A62C 13/66

(52) U.S. Cl. 239/302; 239/318; 239/366

(57) ABSTRACT

A liquid-spraying adapter is provided which connects to a nozzle, which is of a compressed gas supply means of an air gun and the like, and uses the compressed gas thereof to allow a liquid to be sprayed.

A liquid-spraying adapter (2) was constituted, which is a liquid-spraying adapter that may connect to a nozzle (101), which is of a compressed gas supply means (100), provided with a connecting port (4) that may be removably connected to the nozzle (101), a liquid-spraying nozzle port (5) that sprays a liquid, a liquid reservoir coupling port (6) that may be removably coupled to a liquid reservoir (3) that houses a liquid, a communicating passage (7) that communicates between the connecting port (4) and the liquid-spraying nozzle port (5), and a liquid supply passage (8) that communicates between the communicating passage (7) and the liquid reservoir coupling port (6) and may aspirate the liquid inside the liquid reservoir (3) when a compressed gas flows through the communicating passage.

### CROSS-REFERENCE TO RELATED APPLICATIONS

The present application is base on International Application PCT/JP2004/002061 filed on Feb. 23, 2004, and Japanese Patent Applications No. 2003-047933 filed on Feb. 25, 2003, No. 2003-111613 filed on Apr. 16, 2003, No. 2003-111612 filed on Apr. 16, 2003 and No. 2003-180903 filed on Jun. 25, 2003, which applications are incorporated herein by reference in its entirety.

### TECHNICAL FIELD

The present invention relates to a liquid-spraying instrument that is used for spraying various liquids, such as washing solution, coating, paint solution, foamy agent, anti-rust agent, and rust-blackening liquid and, in addition, a variety of oils, a variety of waters, or aqueous solutions. Specifically, the present invention relates to a liquid-spraying adapter that connects to the nozzle of a compressed gas supply means of an air gun and the like and uses the compressed gas thereof to spray a liquid, and to a liquid-supplying liquid reservoir that provides this liquid-spraying adapter with a liquid.

### TECHNICAL BACKGROUND

Currently, in various factories and industrial institutions, such as machinery-manufacturing factories, plastic-forming factories, and automotive repair shops, aerosol containers filled with various liquids are used in various applications. However, while this type of aerosol container allowing a liquid to be sprayed simply and whose portability is also excellent can be used very conveniently, the used aerosol container needs to be disposed of. Moreover, recently, regarding disposal of aerosol containers, a complete elimination of the content is sought, such that, in factories and the like, treatment of used aerosol containers that are generated in large quantities is a problem.

Therefore, the present invention provides, as a novel liquid-spraying instrument that may be substituted for an aerosol container, a liquid-spraying instrument that connects to the nozzle of a compressed gas supply means of an air gun and the like and uses the compressed gas thereof to allow a liquid to be sprayed.

In prior art, a sealed spray gun constituted using a resin accordion container for the coating container of a spray gun for spray-coating use is disclosed (Japanese Patent Application Laid-Open No. 11-28394) as this type of liquid-spraying instrument; however, the constitution is different from the liquid-spraying instrument of the present invention.

### DISCLOSURE OF THE INVENTION

The present inventor focused on a compressed gas supply means of an air gun and the like installed in a number of factories, industrial institutions, and the like, regardless of the type of operation, such as plastic-forming factory and automotive repair factory, and developed a liquid-spraying instrument that directly uses preexisting compressed gas supply means to allow various liquids to be sprayed simply.

That is to say, the liquid-spraying instrument of the present invention is provided with a liquid-spraying adapter that connects to the nozzle of a compressed gas supply means of an air gun and the like and uses the compressed gas thereof to allow a liquid to be sprayed, and a liquid-supplying liquid reservoir that provides the liquid-spraying adapter with a liquid.

The above liquid-spraying adapter is characterized by the provision of a connecting port that may be removably connected to the nozzle of a compressed gas supply means, a liquid-spraying nozzle port that sprays a liquid, a liquid reservoir coupling port that may removably couple a liquid-supplying liquid reservoir housing a liquid, a communicating passage that communicates between the connecting port and the liquid-spraying nozzle port, and a liquid supply passage that communicates between the communicating passage and the liquid reservoir coupling port, which may aspirate the liquid inside the liquid-supplying liquid reservoir when compressed gas flows inside the communicating passage.

The above liquid-supplying liquid reservoir (also referred to as “liquid reservoir”) is provided with a liquid reservoir portion that is able to house a liquid and expandable and contractible and a coupling port/liquid supply port, and is characterized by the provision of a constitution wherein, when the interior of the liquid reservoir portion is aspirated via the coupling port/liquid supply port, the internal liquid is sent out from the coupling port/liquid supply port while the liquid reservoir portion contracts.

As the liquid-spraying instrument of the present invention is provided with the above-mentioned constitution, it can spray various liquids simply by connecting to the nozzle of a compressed gas supply means of an air gun and the like that are already installed in most factories and industrial institutions and can be used extremely inexpensively and simply. Moreover, it can be used permanently if a liquid is supplied to the liquid reservoir. In addition, since it is also possible to spray different liquids by exchanging the liquid reservoir, the problem of disposal treatment of the aerosol container can also be solved. Furthermore, since compressed gas from a compressed gas supply means, such as an air gun is used, it can be used in broader applications compared to an aerosol container, e.g., liquid can be sprayed more strongly compared to an aerosol container.

Note that, in the present invention, “compressed gas supply means” intends to include any apparatus or tool that supplies compressed gas (also referred to as “compressed gas supply apparatus or tool”), such as a compressor, a pump, an air gun, and a spray gun, and the “nozzle” thereof is the front extremity of a spraying tool that sprays a compressed gas supplied by the compressed gas supply apparatus or tool, for instance, the nozzle and the like of an air gun and so on.

An “air gun” is a spraying tool that sprays compressed air from a compressor and the like, is, in general, provided with a grip, a trigger, a nozzle, an air nipple, and the like, and is arranged in such a way that compressed air is sent in from the air nipple, and the compressed air can be sprayed from the nozzle by operating the trigger. The so-called spray gun, which is provided with a nipple that connects to a container that houses a liquid, such as coating, or to a hose, atomizes a liquid, such as coating, by blowing the compressed air out to spray a liquid from the nozzle, is also a type of air gun; however, in this case, it must be provided with a structure that allows only compressed air to be sprayed from the nozzle via a switch valve or the like.

In the present invention, “liquid” includes a variety of liquids used in any application, regardless of the form, such as washing solution, coating, paint solution, foamy agent, anti-rust agent, and rust-blackening liquid and, in addition, various oils, various waters, or aqueous solutions.

“May (removably) connect to the nozzle of a compressed gas supply means” includes all connective structures that may couple without leakage of compressed gas and includes, additionally, for instance, a structure that connects threadably and a structure that connects by fitting, and, for instance, a structure that connects by pressing the nozzle of a compressed gas supply means against the connecting port so that the compressed gas does not leak out, and the like.

### BRIEF DESCRIPTION OF THE DRAWINGS

FIG. 1 is a side view showing an example of a liquid-spraying adapter according to one example of the present invention, disassembled into an adapter main body and a liquid reservoir, together with an air gun.

FIG. 2 is a cross-sectional view of the adapter main body shown in FIG. 1.

FIG. 3 is a cross-sectional view showing an enlargement of the relevant portion of the adapter main body to show an example of variation of the small-diameter passage of a communicating passage.

FIG. 4 is a cross-sectional view showing the flow of compressed gas inside a communicating passage together with an enlargement of the relevant portion of the adapter main body to describe the location where the liquid supply passage couples to the communicating passage.

FIG. 5 is a cross-sectional view showing an enlargement of the relevant portion of the adapter main body to show an example of variation of a coupling liquid supply passage.

FIG. 6 is a side view of the liquid reservoir shown in FIG. 1.

FIG. 7 is a side view explaining the action of the liquid reservoir shown in FIG. 6.

FIG. 8 is a partial cross-sectional side view showing a method for using an example of a liquid-spraying adapter according to one example of the present invention.

FIG. 9 is a side view showing one example of a liquid-supplying liquid reservoir according to the present invention, one example of a liquid-spraying adapter, and an air gun as one example of a compressed gas supply means, in a disassembled state.

FIG. 10 is a cross-sectional view showing an enlargement of the relevant portion of the coupling portions of a liquid-supplying liquid reservoir and a liquid-spraying adapter, in a disassembled state.

FIG. 11 is a cross-sectional view showing an enlargement of the relevant portions of the coupling portions of a liquid-supplying liquid reservoir and a liquid-spraying adapter, in an assembled state.

FIG. 12 is a cross-sectional view showing an enlargement of the relevant portion of a constitutional example of a coupling port/liquid supply port of a liquid-supplying liquid reservoir, in a disassembled state.

FIG. 13 is a perspective view showing an enlargement of the relevant portion of a constitutional example of a coupling port/liquid supply port of a liquid-supplying liquid reservoir, in a disassembled state.

FIG. 14 is a partial cross-sectional view showing the liquid-supplying liquid reservoir and the liquid-spraying adapter shown in FIG. 9 in use.

FIG. 15 is a side view of an example of variation of a liquid-supplying liquid reservoir.

FIG. 16 is a top view of the liquid-supplying liquid reservoir shown in FIG. 15.

FIG. 17 is a side view of one usage example of the liquid-supplying liquid reservoir shown in FIG. 15.

FIG. 18 is a cross-sectional view showing one example of the liquid-spraying adapter of the present invention.

FIG. 19 is a side view showing one example of the liquid-supplying liquid reservoir and the liquid-spraying adapter, developed by the present inventor and undisclosed at the time of filing the present application, and an air gun as one example of a compressed gas supply means, in a disassembled state.

FIG. 20 is a cross-sectional view showing an example of variation of a liquid reservoir.

FIG. 21 is a partial cross-sectional side view of the coupling structure of the liquid reservoir shown in FIG. 20 and a liquid-spraying adapter.

FIG. 22 is a cross-sectional view showing an example of variation of a liquid reservoir.

FIG. 23 is a cross-sectional view showing one usage example of the liquid reservoir shown in FIG. 22.

FIG. 24 is a cross-sectional view showing one constitutional example of the liquid reservoir shown in FIG. 23.

FIG. 25 shows the front, left and right sides, and top and bottom views of a liquid-spraying adapter, which is a constitutional element of another constitutional example of the liquid-spraying adapter of the present invention.

FIG. 26 is a cross-sectional view along the A-A line in FIG. 25.

FIG. 27 is an external view cross-sectionally showing half of the surface of a liquid reservoir, which is a constitutional element of another constitutional example of a liquid-spraying adapter of the present invention.

FIG. 28 is a cross-sectional view showing an enlargement of a coupling portion when the liquid reservoir of FIG. 27 is mounted on the liquid-spraying adapter of FIG. 25.

### EMBODIMENT

In the following, an embodiment of the present invention will be described; however, the present invention is not limited to this embodiment.

The liquid-spraying instrument of the present invention consists of a liquid-spraying adapter and a liquid-supplying liquid reservoir, and each can be formed as shown below.

Liquid-Spraying Adapter

The liquid-spraying adapter of the present invention, as mentioned above, is provided with a connecting port that may be removably connected to the nozzle of a compressed gas supply means, a liquid-spraying nozzle port that sprays a liquid, a liquid reservoir coupling port that may removably couple a liquid-supplying liquid reservoir housing a liquid, a communicating passage that communicates between the connecting port and the liquid-spraying nozzle port, and a liquid supply passage that communicates between the communicating passage and the liquid reservoir coupling port.

In the above constitution, “communicating passage and liquid supply passage” preferably has a constitution wherein, when compressed gas flows inside the communicating passage, the liquid inside the liquid reservoir is aspirated via the liquid supply passage into the communicating passage. The concrete structure thereof can be formed arbitrarily, for instance, the constitution is such that a step portion is provided in the intermediate portion of the communicating passage, the connecting port side of the step portion being a small-diameter passage, the liquid-spraying nozzle port side of the step portion being a large-diameter passage whose diameter is larger than the small-diameter passage, and the liquid supply passage establishing a communication in the vicinity of the step portion within the large-diameter passage. With such a structure, the liquid inside the liquid reservoir can be aspirated inside the communicating passage via the liquid supply passage by flowing a compressed gas inside the communicating passage, allowing the liquid to be sprayed from the liquid-spraying nozzle port.

Note that, in so doing, the vicinity of the step portion, as described below, means a range such that, when the compressed gas flown through the small-diameter passage enters the large-diameter passage and diffuses, a negative pressure is generated by the diffusion.

A spray adjustment tool that may adjust the spraying amount, the spray shape, and the like can be connected to the liquid-spraying nozzle port of the liquid-spraying adapter provided with the above constitution. Since the spraying amount, the spray shape, and the like can be adjusted with the spray adjustment tool, it becomes even easier to use, further expanding the application.

In addition, a selector switch that may open and close the liquid supply passage can be provided, and if such a selector switch is provided, spraying of liquid and spraying of high-pressure gas can be switched freely, allowing workability to be further increased.

Liquid-Supplying Liquid Reservoir

The liquid-supplying liquid reservoir (also referred to as “liquid reservoir”) is coupled to the liquid reservoir coupling port of the liquid-spraying adapter provided with the above-mentioned constitution. Although not to limit the structure thereof in particular, for instance, preferred are those provided with a constitution in which a liquid reservoir portion that is capable of housing a liquid and is expandable and contractible and a coupling port/liquid supply port (also referred to as “coupling port”) are provided, and when the interior of the liquid reservoir portion is aspirated from the coupling port, the liquid inside is sent out from the coupling port while the liquid reservoir portion contracts.

If the liquid reservoir is provided with such a constitution, the housed liquid can be sprayed from the liquid-spraying nozzle port via the communicating passage of the liquid-spraying adapter simply by coupling to the liquid reservoir coupling port of the liquid-spraying adapter and passing the compressed gas from the compressed gas supply means into the communicating passage. Moreover, since the structure is such that the liquid reservoir main body contracts when aspiration occurs, the liquid reservoir main body need not be provided with an air vent; therefore, the liquid can be sprayed equally, should the liquid reservoir be used at any angle, which can be used without being upside-down.

In the liquid reservoir provided with the above-mentioned constitution, preferably, the opening of the coupling port is openably closed.

If the liquid reservoir is carried around in a state where a liquid is housed, as the liquid reservoir portion is expandable and contractible, there is the possibility that the liquid reservoir portion contracts, and the liquid leaks out from the coupling port. In addition, when coupling the liquid reservoir to the liquid supply adapter, unless performed considerably cautiously, there is the possibility that the liquid reservoir portion contracts, and the liquid leaks out from the coupling port. Therefore, if the opening of the coupling port is openably closed, as described above, since the opening of the coupling port is closed in the normal state, the liquid does not leak out, even if the liquid reservoir portion contracts, while by opening the blockage as necessary, the liquid can be supplied to the liquid supply adapter. Also, volatilization of the liquid can also be prevented. Further, with the opening of the coupling port in the closed state, if, for instance, the liquid reservoir is coupled to the liquid supply adapter, and compressed gas is sprayed from the air gun, flowing only compressed gas through the interior of the communicating passage and the liquid supply passage in the liquid supply adapter becomes possible, allowing the interior of the communicating passage and the liquid supply passage to be flushed.

The structure for openably closing the opening of the coupling port is not limited in particular. For instance, the opening can be openably closed by closing with a plastic sheet, film, or the like (namely, by covering with a thin part). In so doing, while the plastic sheet, film, or the like can be formed into one body with the liquid reservoir, the constitution can also be a separate body of plastic sheet, film, or the like covering the opening.

In addition, the coupling port can also be constituted in such a way that it may removably attach a cap body whose top surface is formed to be openable, for instance, a cap body whose top surface thickness is thinly formed to be openable, and the opening of the coupling port can be openably closed by wearing the cap body. However, since the constitution must be such that the reservoir may couple with the liquid reservoir coupling port of the liquid-spraying adapter in a state where the cap body is worn, provision of a structure coupling with the liquid reservoir coupling port of the liquid-spraying adapter, such as, for instance, provision of a screw portion on the outer peripheral surface of the cap body, is necessary.

If the constitution is such that a cap body formed with an openable top surface, as described in the foregoing, is worn, since the blockage of the opening can be re-created by replacing the cap body, the main body of the liquid reservoir can be used repeatedly. In addition, when the use has the purpose of flushing the interior of the adapter, as described above, since all that is needed is to wear the cap body, it is extremely simple.

Note that, although a liquid reservoir having such a constitution can also be used by coupling with a liquid-spraying adapter provided with a liquid reservoir coupling port having no means for opening the blockage of the coupling port on the liquid reservoir, from the viewpoint of preventing the leakage at the time of coupling, coupling with a liquid-spraying adapter provided with means for opening the blockage of the coupling port on the liquid reservoir is preferred. Specifically, a liquid-spraying adapter, wherein inside the liquid reservoir coupling port is provided a pointed tubular projection having a liquid supply passage on the inside, is preferred. In so doing, the tubular projection is preferably provided inside the coupling depression.

If the liquid reservoir of the present invention is coupled to the liquid reservoir coupling port of a liquid-spraying adapter provided with such a constitution, since the coupling port of the liquid reservoir is closed until coupling time, the liquid does not leak out; moreover, since the blockage of the coupling port of the liquid reservoir can be opened by tearing with the tubular projection simultaneously to coupling, the liquid can be rendered supplyable to the liquid-spraying adapter.

### EXAMPLES

In the following, examples of the present invention will be described based on figures; however, the present invention is not limited to these examples.

### Example 1

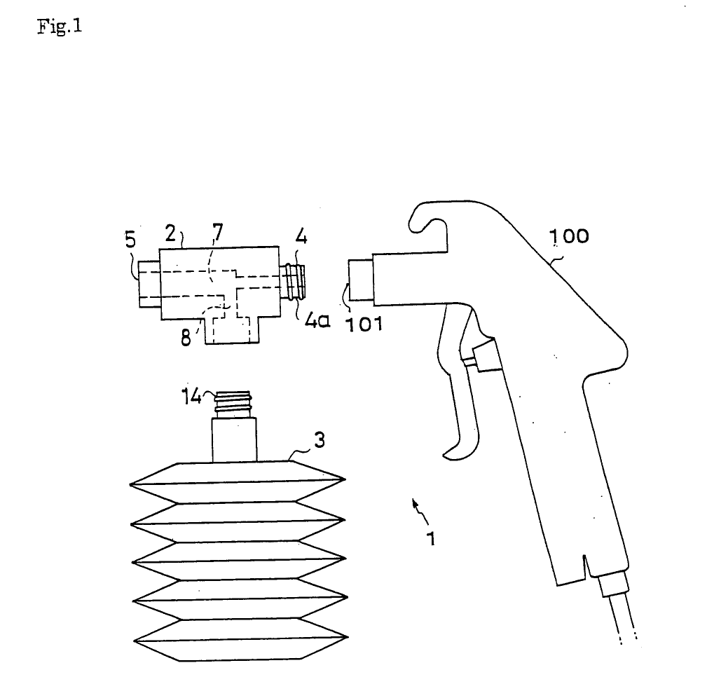

A liquid-spraying instrument 1 according to an example of the present invention was an instrument that may connect to a nozzle 101 of an air gun 100 and is provided with a liquid-spraying adapter 2 and a liquid reservoir 3, as shown in FIG. 1.

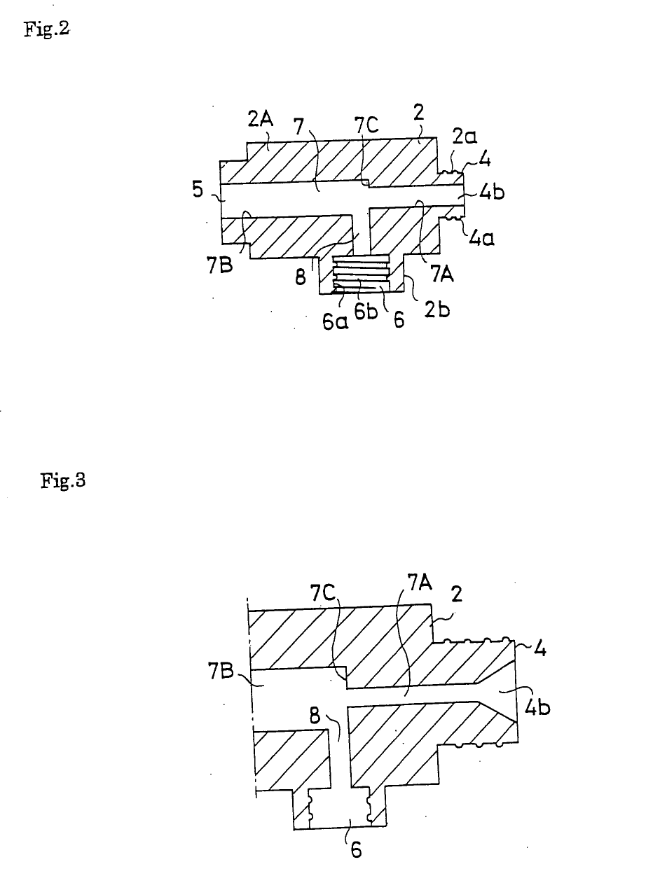

The liquid-spraying adapter 2, as shown in FIG. 2, was provided with a connecting port 4 capable of being removably connected to the nozzle 101 of the air gun 100, a liquid-spraying nozzle port 5 for spraying a liquid, a liquid reservoir coupling port 6 capable of being removably coupled to a liquid reservoir 3, a communicating passage 7 for communicating between the connecting port 4 and the liquid-spraying nozzle port 5, and a liquid supply passage 8 for communicating between the communicating passage 7 and the liquid reservoir coupling port 6.

The material of the adapter main body portion 2A that constitutes the main body of the liquid-spraying adapter 2 is not limited in particular and can be formed, for instance, from a synthetic resin, such as thermoplastic resin and thermosetting resin, or metal, rubber, elastomer (a resin provided with elasticity), and the like.

Molding into one body from a synthetic resin, a metal or the like is also possible; however, there is no limitation to molding into one body. In the case of the present example, a synthetic resin was injection molded, and in the thick-walled prismatic main body portion 2A, the connecting port 4, the liquid-spraying nozzle port 5, the liquid reservoir coupling port 6, the communicating passage 7, and the liquid supply passage 8 were molded into one body. However, one or two or more among the connecting port 4, the liquid-spraying nozzle port 5, the liquid reservoir coupling port 6, the communicating passage 7, and the liquid supply passage 8 can be formed from a material different from the main body portion 2A and subsequently fixed onto the main body portion 2A to form into one body.

Note that the shape of the main body portion 2A is arbitrary, and forming can be into rectangular shape, cylindrical shape, substantially conical shape, rectangular tubular shape, round tubular shape, or any other shape.

The connecting port 4 is formed in such a way that it can couple to the nozzle 101 of the air gun 100 in a manner that is removable and may retain airtightness. For instance, in the case of the present example, it was constituted by providing a projection 2a from the main body portion 2A, forming on the outer peripheral surface of the projection 2a a screw portion 4a capable of being screwed into the screw portion provided in the inner peripheral surface of the nozzle 101, at the same time providing inside the projection 2a an opening 4b for communicating with the communicating passage 7.

As shown in FIG. 2, the liquid-spraying nozzle port 5 may have a structure that opens directly with the size of the communicating passage 7 (specifically, a large-diameter passage 7B, described below) or can be formed otherwise at will. For instance, it can be formed by varying adequately the shape and size of the opening thereof according to a shape for spraying a liquid. In addition, a diaphragm mechanism that allows the opening to be opened and closed at will can also be provided, such that the spraying amount, the spray shape, and the like may be adjusted. In addition, it can be formed to be connectable with a spray adjustment tool that may adjust the spraying amount, the spray shape, and the like, and other adapters, for instance, by forming a screw portion on the inner peripheral surface or the outer peripheral surface of the opening and the like.

The liquid reservoir coupling port 6 was engraved by providing a projection 2b on the bottom surface of the main body portion 2A, providing a depression 6a inside the projection 2b into which the coupling port 14 may insert, and providing on the inner peripheral surface of this depression 6a a screw portion 6b capable of being screwed onto the screw portion of the coupling port 14. However, any coupling structure that may removably couple to the coupling port 14 of the liquid reservoir 3 while retaining airtightness can be adopted. For instance, it can be formed by embedding in the depression 6a a cap body provided with a screw portion on the inner peripheral surface.

The communicating passage 7 was formed as a hollow space for communicating between the connecting port 4 and the liquid-spraying nozzle port 5. Specifically, as shown in FIG. 2, the communicating passage 7 was formed by providing a step portion 7C at an intermediate portion, the connecting port 4 side of this step portion 7C being a small-diameter passage 7A consisting of a hollow space with a smaller diameter, and the liquid-spraying nozzle port 5 side of the step portion 7C being the large-diameter passage 7B consisting of a hollow space with a larger diameter.

In so doing, the size of the step portion 7C, namely, the difference in the diameters between the small-diameter passage 7A and the large-diameter passage 7B, is preferably approximately 5 mm or more.

The small-diameter passage 7A may be formed, as shown in FIG. 3, by narrowing into a small diameter from the opening 4b of the connecting port 4.

The liquid supply passage 8 was constituted in such a way that it communicated between the communicating passage 7 and the liquid reservoir coupling port 6 approximately perpendicularly to the communicating passage 7. Specifically, the liquid supply passage 8 establishes a communication in the vicinity of the step portion 7C within the large-diameter passage 7B, and when a compressed gas flows inside the communicating passage 7, the pressure inside the liquid supply passage 8 becomes negative, and the liquid inside the liquid reservoir 3 may be aspirated.

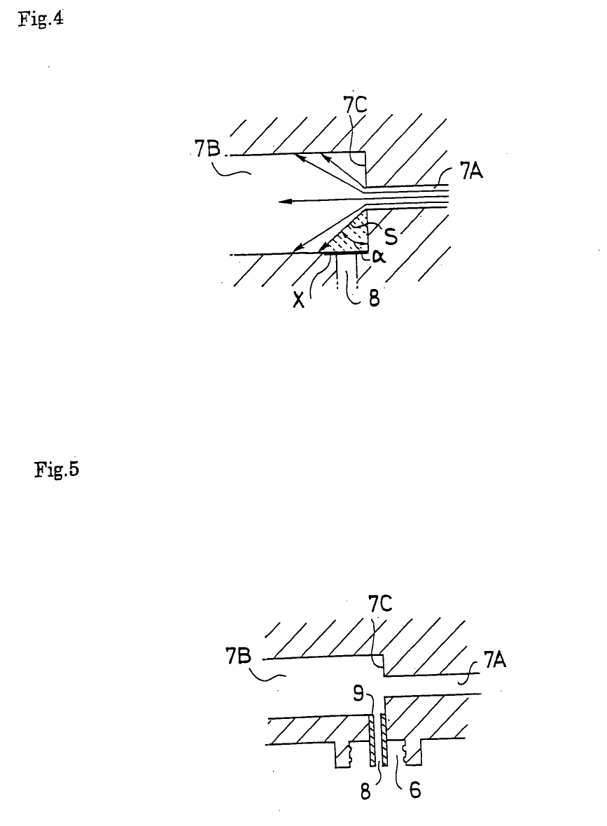

Note that, in the case of the present example, communication was established in such a way that the step surface of the step portion 7C and the inner peripheral surface on the connecting port 4 side of the liquid supply passage 8 were flush, and communication was established within a vicinity range (thick line portion X in the figure) from the step portion 7C as shown in FIG. 4. That is to say, when a compressed gas flowing through the small-diameter passage 7A enters the large-diameter passage 7B and diffuses at once, the inside corner area (broken line portion) of this diffusion line S is the range where the pressure becomes negative, and if the liquid supply passage 8 is establishing a communication in the vicinity range (thick line portion X in the figure), the pressure inside the liquid supply passage 8 becomes negative when the compressed gas flows inside the communicating passage 7, and the liquid inside the liquid reservoir 3 can be aspirated.

In so doing, assuming that the angle a of the diffusion line S is 45 degrees, the vicinity range (thick line portion X in the figure) can be set as the portion that is more towards the step portion 7C than the position where the diffusion line S intersects the inner peripheral surface of the large-diameter passage 7B.

In other words, to form the adapter main body portion 2A in such a way that the liquid inside the liquid reservoir 3 is aspirated into the communicating passage 7 when a compressed gas is flown through the communicating passage 7, a constitution wherein the diameter of the communicating passage 7 is made larger than the liquid-spraying nozzle port 5 side via the step portion 7C while the liquid supply passage 8 is coupled in the vicinity of the step portion 7C within the large-diameter passage 7B of the communicating passage 7 having an expanded diameter, is sufficient.

In so doing, the angle at which the liquid supply passage 8 intersects the communicating passage 7 is not limited in particular, and forming an intersection that is approximately perpendicular, as in the present example, or additionally, forming an intersection that is inclined at any angle is also possible.

The size of the diameter of the liquid supply passage 8 is preferably designed to be an appropriate size that is adequate to aspirate the liquid inside the liquid reservoir 3. In general, for instance, when the diameter of the large-diameter passage 7B is on the order of approximately 4 mm, the internal diameter of the liquid supply passage 8 is preferably on the order of approximately 1.5 mm. However, the size of the diameter is not limited thereto.

Further, additionally, as shown in FIG. 5, the liquid supply passage 8 can also be formed by perforating a hole in the main body portion 2A and pushing a tube member 9 inside the hole, in which case, the liquid supply passage 8 can also be formed in such a way that the tip of the tube member 9 is made to protrude inside the liquid reservoir coupling port 6, and when the coupling port 14 of the liquid reservoir 3 is coupled to the liquid reservoir coupling port 6, the tip of the tube member 9 penetrates into the coupling port 14.

As shown in FIG. 6, the liquid reservoir 3 was constituted by a liquid reservoir main body 11 that was capable of retaining a liquid and was expandable and contractible.

This liquid reservoir main body 11 was formed from a synthetic resin and was constituted by a body portion 12 that was formed into a retractable accordion shape, a circular tube portion 13 protruding above the body portion 12, and a coupling portion 14 that was formed at the upper end portion of the circular tube portion 13 and was capable of being coupled removably to a liquid reservoir coupling port 6.

The coupling portion 14 was formed to be screwable to the screw portion 6b of the liquid reservoir coupling port 6.

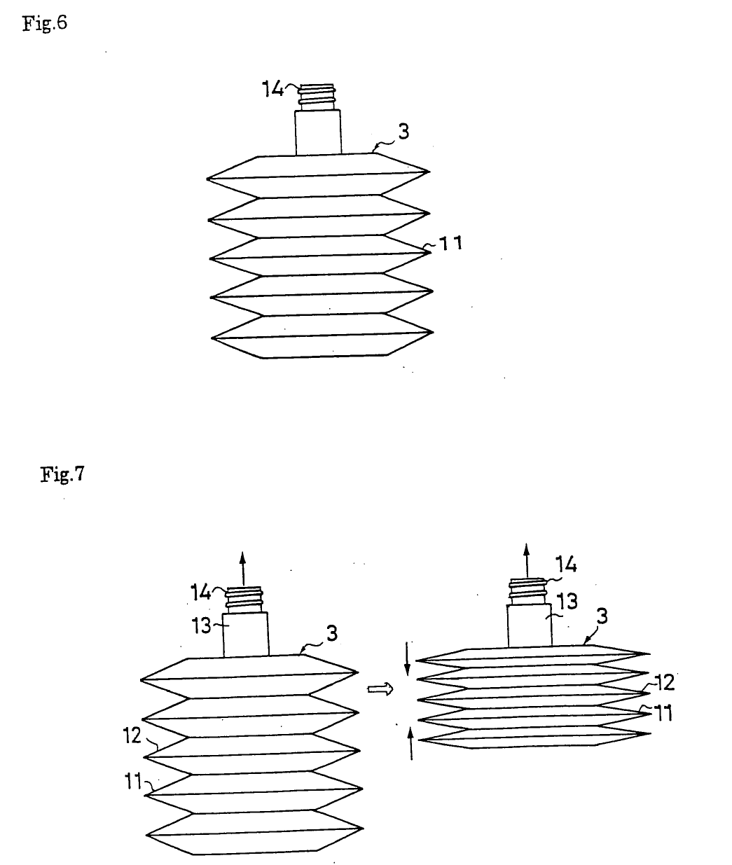

Such a liquid reservoir 3, when coupled to the liquid reservoir coupling port 6, the state inside the body portion 12 becomes sealed, but since it is expandable and contractible, when aspirating from the coupling portion 14 side in a state where a liquid is housed inside the liquid reservoir main body 11, the liquid inside flows out from the mouth of the coupling portion 14 while the body portion 12 of the liquid reservoir main body 11 contracts, as shown in FIG. 7.

In addition, although not shown, since the liquid reservoir 3 has no air vent, it can provide the liquid-spraying adapter 2 with a liquid equally, should it be used at any angle spanning 360°, for instance, even when used upside down from the state in FIG. 7.

Note that, with a constitution wherein a removable cap is worn by the coupling portion 14, the liquid reservoir 3 can also be used as a liquid storage container when not in use.

In the following, a method for using the liquid-spraying instrument 1 will be described.

The liquid-spraying instrument 1 was assembled by mounting the liquid reservoir 3 housing a liquid to the liquid-spraying adapter 2 by coupling the coupling portion 14 thereof to the liquid reservoir coupling port 6 of the liquid-spraying adapter 2.

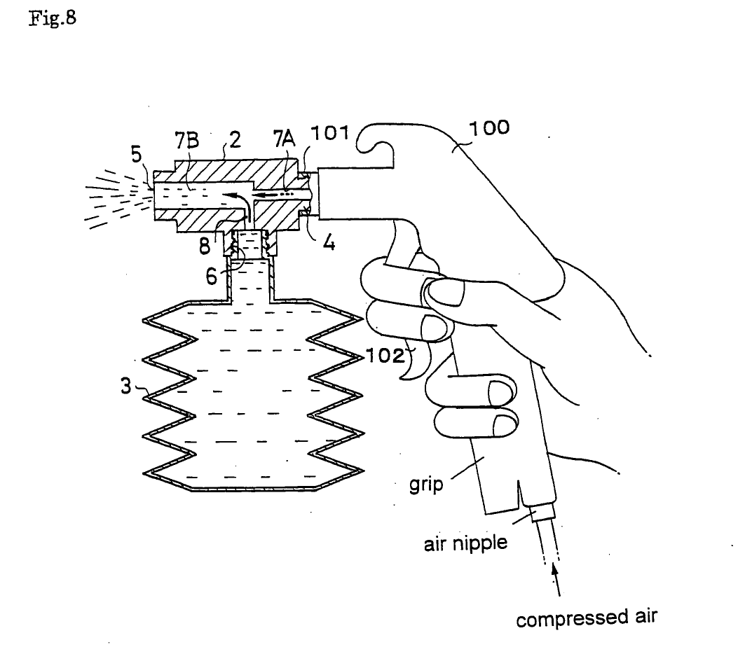

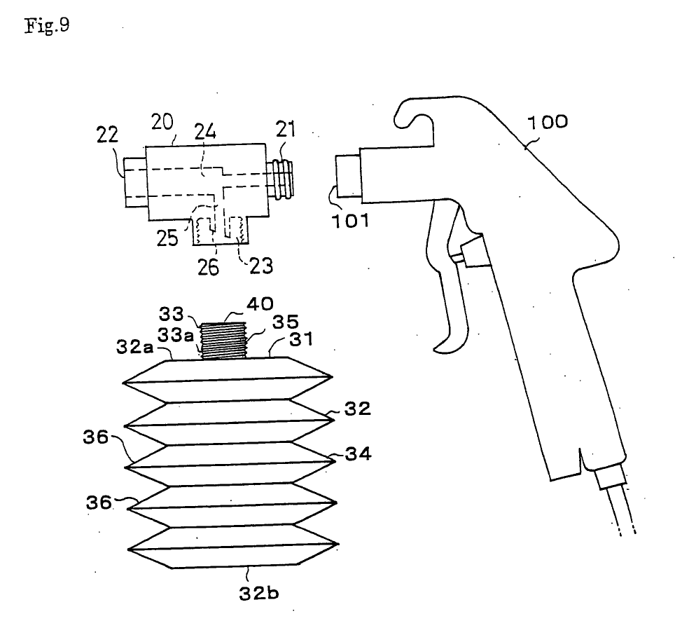

Then, it is ready to spray when the connecting port 4 is connected to the nozzle 101 of the air gun 100, as shown in FIG. 8.

Thereafter, the compressed gas (compressed air) can be sprayed from the nozzle 101 by operating the trigger 102 of the air gun 100. In so doing, the compressed gas flows through the communicating passage 7 and blows out from the liquid-spraying nozzle port 5 while aspirating the liquid inside the liquid reservoir 3, making the liquid blow out from the liquid-spraying nozzle port 5.

In this way, by the use of the liquid-spraying instrument 1 of the present invention, a liquid can be continuously sprayed using a compressed gas supply apparatus or tool, such as an air gun, already installed in factories and other industrial institutions and the like, without having to newly install an apparatus that sends a liquid by pressure; moreover, the liquid can be sprayed equally, should it be used at any angle spanning 360°, namely, without being upside-down.

### Example 2

Example 2 according to a liquid-spraying instrument was constituted by a liquid-spraying adapter 20 and a liquid reservoir 31, as shown in FIG. 9.

Liquid-Supplying Liquid Reservoir

The liquid reservoir 31 was constituted in such a way that a liquid reservoir portion 32 that was capable of housing a liquid, and was expandable and contractible, and a coupling port/liquid supply port 33 (also referred to as “coupling port”) were provided, and when the interior of the liquid reservoir portion 32 was aspirated from the coupling port 33, the liquid inside was sent out from the coupling port 33 while the liquid reservoir portion 32 contracted.

The liquid reservoir 31 can be formed from a synthetic resin, and it suffices to suitably select the type of synthetic resin according to the type of liquid to be housed, with no particular limitation; however, a liquid reservoir portion 32 that may provide some degree of shape retainability is preferred. For instance, polyethylene, polypropylene, polyethylene terephthalate, and the like can be given as preferred examples.

The liquid reservoir portion 32 had a constitution provided with an accordion portion 34 on the side of a circular cylindrical body provided with a top surface 32a and a bottom surface 32b, the accordion portion 34 having a constitution wherein two steps or more (namely, a plurality of steps; five steps in the figure) of an projected portion (in other words, a “crease”) 36, consisting of a mountain-folded portion sandwiched between valley-folded portions, were continuously provided in the top-down direction (namely, the direction that joined the coupling port 33 and the bottom surface 32b).

Each projected portion 36 was such that the mountain folded portion projected in a triangular shape in the outward direction (namely, the direction oriented outward from the center of the liquid reservoir portion 32 in the direction perpendicular to the top-down direction) opened and closed in the top-down direction, and the entire liquid reservoir portion 32 extended and shrunk in the top-down direction.

However, it is preferred that the extent of shape retainability with which the accordion portion 34 is provided is such that the accordion portion 34 does not extend in the top-down direction, even when a liquid is housed inside.

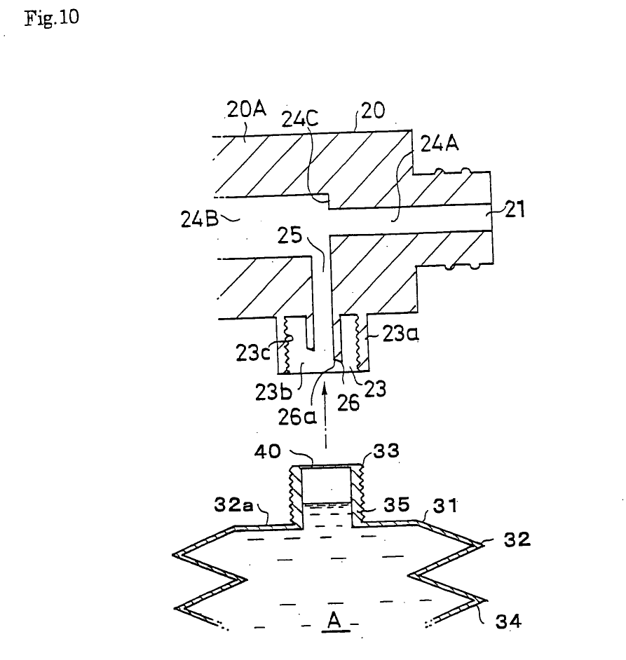

As shown in FIG. 10, the coupling port 33 was constituted in such a way that a circular tube portion 35 was protruding from the top surface 32a of the liquid reservoir portion 32, and the thickness of the top surface of this circular tube portion 35 was thinned to create an openable blockage 40.

While the blockage 40 can be molded into one body with the liquid reservoir portion 32 and the like such that the thickness becomes thin, it can also be constituted in such a way that a plastic sheet, film, or the like, which is a separate body, covers the opening.

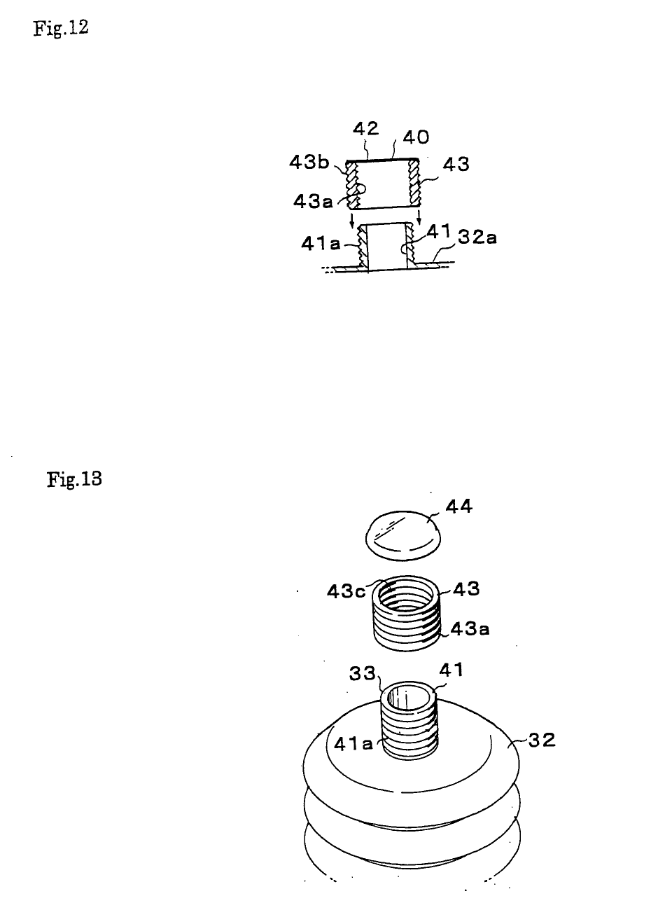

Preferably, as shown in FIG. 12, the coupling port 33 is constituted, on one hand, by protruding a circular tube portion 41 from the top surface 32a of the liquid reservoir portion 32 and providing a screw portion 41a on the outer peripheral surface of the circular tube portion 41, and on the other hand, by preparing, as a cap that is a separate body therefrom, a circular cylindrically shaped cap body 43 that can be removably mounted on the outside of this circular tube portion 41 and whose top surface 42 was formed to have a thin thickness and by mounting the cap body 43 to the above-mentioned circular tube portion 41.

In so doing, it is preferred that the cap body 43 is provided with a screw portion 43a on the inner peripheral surface thereof, which is screwable with the screw portion 41a of the above-mentioned circular tube portion 41, and that it is also provided with a screw portion 43b on the outer peripheral surface thereof.

Note that, while the top surface 42 of the cap body 43 can be molded into one body with the peripheral portion and the like such that only the thickness of the top surface 42 is thin, it can also be constituted in such a way that a plastic sheet, film, or the like 44, which is a separate body from the cap body 43, covers the opening 43c of the cap body 43, as shown in FIG. 13.

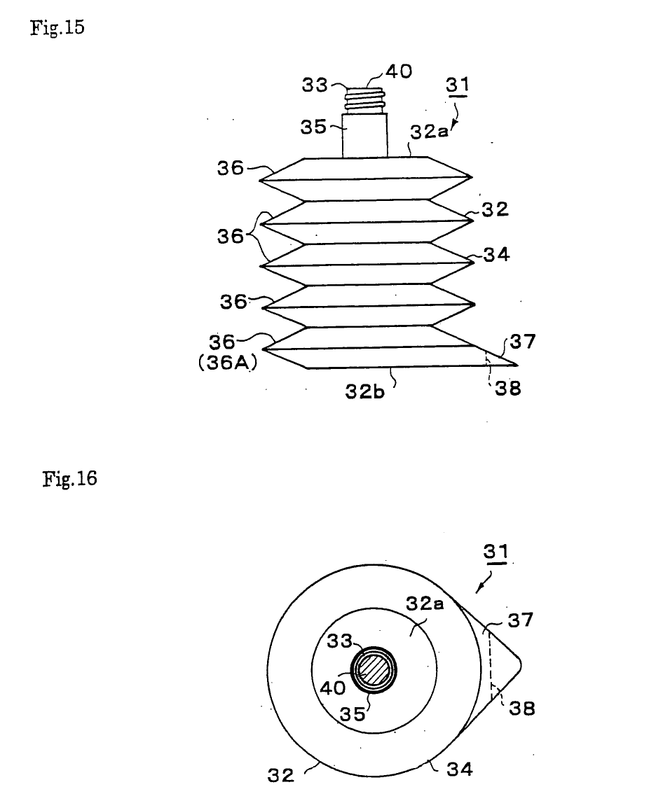

As shown in FIG. 15 to FIG. 17, the projected portion 36A that is at the bottommost position among a plurality of projected portions 36 constituting the accordion portion 34 may have a portion, specifically, a portion of the surrounding edge protruding outwards in the radial direction, to provide a protrusion 37. In so doing, the shape of the protrusion 37 can be formed into a triangular shape, a circular arc shape, a square shape, and any other shape, when the shape is viewed from the top surface.

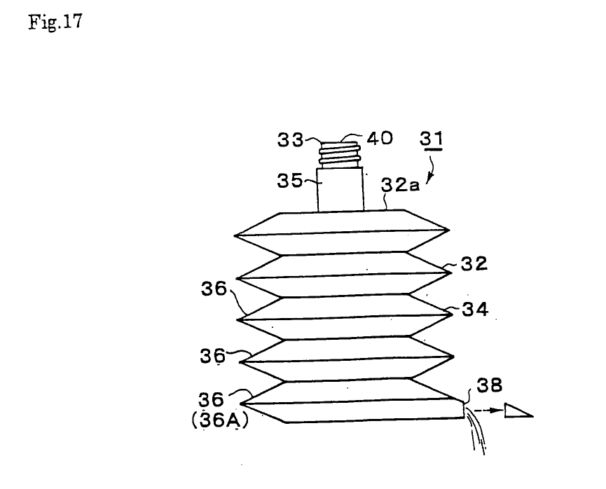

If a protrusion 37 is provided in this way to the projected portion 36A, as shown in FIG. 17, by cutting the adequate intermediate portion of protrusion 37, the liquid left inside can be easily eliminated, allowing the liquid reservoir 31 to be disposed of in a state where the liquid has been completely eliminated.

Note that for cutting the protrusion 37, although it suffices to cut using scissors, a cutter, or the like, a cut line 38 can also be formed beforehand to make cutting easy. For instance, it is possible to form the cut line 38 with a thin thickness to make cutting easy.

In addition, cutting of the protrusion 37 can be such that the proximal extremity of the protrusion 37 is completely cut off, as shown in FIG. 17; however, it can be nicked to such an extent that the liquid can be eliminated, without completely cutting it off.

Liquid-Spraying Adapter

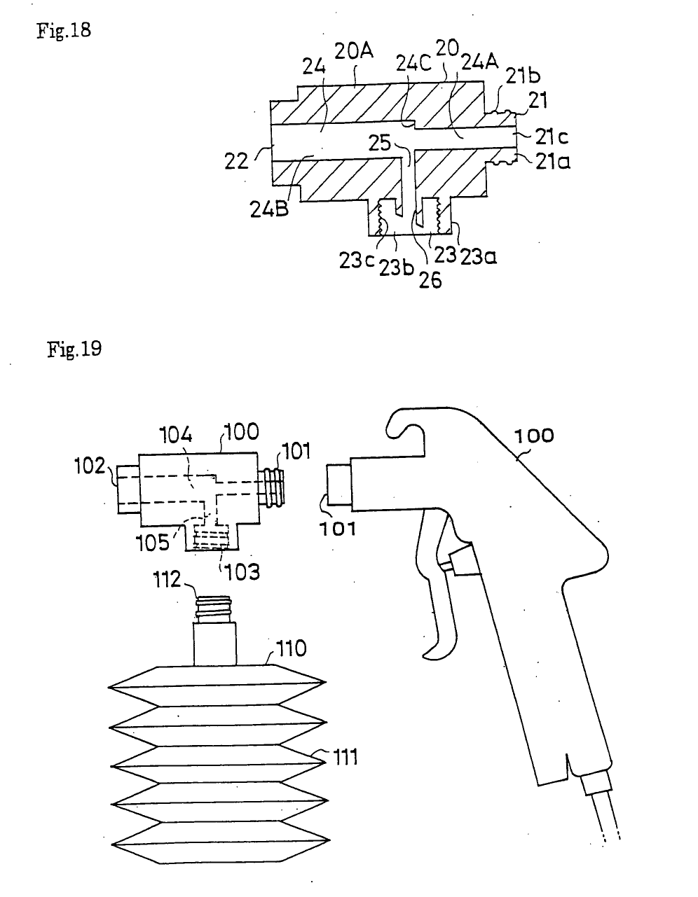

The liquid-spraying adapter 20 was provided with a connecting port 21 capable of being removably connected to the nozzle 101 of the air gun 100, a liquid-spraying nozzle port 22 for spraying a liquid, a liquid reservoir coupling port 23 capable of being removably coupled to the liquid reservoir 31, a communicating passage 24 for communicating between the connecting port 21 and the liquid-spraying nozzle port 22, and a liquid supply passage 25 for communicating between the communicating passage 24 and the liquid reservoir coupling port 23, as shown in FIG. 9 and FIG. 18.

The material of the adapter main body 20 that constitutes the main body of the liquid-spraying adapter 20 is not limited in particular and can be formed, for instance, from a synthetic resin, such as thermoplastic resin and thermosetting resin, or metal, rubber, elastomer (a resin provided with elasticity), and the like.

Molding into one body from a synthetic resin, a metal or the like is also possible; however, there is no limitation to molding into one body.

The connecting port 21 was formed in such a way that it could couple to the nozzle 101 of the air gun 100 in a manner that was removable and capable of retaining airtightness. For instance, in the case of the present example, the connecting port 21 was constituted by providing the adapter main body 20 with a circular tubular projection 21a, forming on the outer peripheral surface of the projection 21a a screw portion 21b capable of being screwed into the screw portion provided on the inner peripheral surface of the nozzle 101, and providing inside the projection 21a an opening 21c for communicating with the communicating passage 24.

However, if the connecting port 21 can couple to the nozzle 101 of the air gun 100 in a manner that is removable and may retain airtightness, the connecting structure thereof can be formed arbitrarily. For instance, although not shown, it can also have a constitution wherein a nozzle contact opening is provided with a tapered surface that continuously opens outwards from the open extremity of the communicating passage 24, allowing the tip (nozzle 101) of the air gun 100 to be in contact with the taper face in an approximately airtight state.

The liquid-spraying nozzle port 22 may have a structure that opens directly with the size of the communicating passage 24 (specifically, a large-diameter passage 24B, described below) or can be formed otherwise at will. For instance, it can be formed by varying adequately the shape and size of the opening thereof, according to the shape for spraying the liquid. In addition, a diaphragm mechanism that allows the opening to be opened and closed at will can also be provided, such that the spraying amount, the spray shape, and the like may be adjusted. In addition, it can be formed to be connectable with a spray adjustment tool that may adjust the spraying amount, the spray shape, and the like, and other adapters, for instance, by forming a screw portion on the inner peripheral surface or the outer peripheral surface of the opening and the like.

The liquid reservoir coupling port 23 was provided by providing a projection 23a on the bottom surface of the adapter main body 20A, providing a coupling depression 23b that opened downwards inside the projection 23a, and providing a screw portion 23c on the inner peripheral surface of this coupling depression 23b, while protruding a tubular projection 26 at a central portion inside the coupling depression 23b, as shown in FIG. 10 and FIG. 18.

The tubular projection 26 had a tip 26a formed into a tapered shape (pointed shape) to allow the blockage 40 of the coupling port 33 of the liquid reservoir 31 to be punched through, the liquid supply passage 25 passing inside and opening at the tip 26a.

The communicating passage 24 was formed as a hollow space for communicating between the connecting port 21 and the liquid-spraying nozzle port 22. Specifically, as shown in FIG. 10 and FIG. 18, it was formed by providing a step portion 24C at an intermediate portion, the connecting port 21 side of this step portion 24C being a small-diameter passage 24A, consisting of a hollow space with a smaller diameter, and the liquid-spraying nozzle port 22 side of the step portion 24C being a large-diameter passage 24B, consisting of a hollow space with a larger diameter.

In so doing, the size of the step portion 24C, namely, the difference in the diameters between the small-diameter passage 24A and the large-diameter passage 24B, is preferably approximately 5 mm or more.

Note that the small-diameter passage 24A may be formed by narrowing into a small diameter from the opening 21c of the connecting port 21.

The liquid supply passage 25 was constituted in such a way that it communicated between the communicating passage 24 and the liquid reservoir coupling port 23 approximately perpendicularly to the communicating passage 24. Specifically, the liquid supply passage 25 establishes a communication in the vicinity of the step portion 24C within the large-diameter passage 24B, and when a compressed gas flows inside the communicating passage 24, the pressure inside the liquid supply passage 25 becomes negative, and the liquid inside the liquid reservoir 31 may be aspirated.

Note that, although in the case of the figure, communication is established in such a way that the step surface of the step portion 24C and the inner peripheral surface on the connecting port 21 side of the liquid supply passage 25 are flush, communication can be established within a vicinity range from the step portion 24C even if they are not flush. That is to say, when a compressed gas flowing through the small-diameter passage 24A enters the large-diameter passage 24B and diffuses at once, if the liquid supply passage 25 is establishing a communication within a range where the pressure in the inside corner area of the step portion 24C becomes negative (the previously described vicinity range), the pressure inside the liquid supply passage 25 can be made to be negative when the compressed gas flows inside the communicating passage 24, allowing the liquid inside the liquid reservoir 31 to be aspirated.

In other words, to form the adapter main body portion 2A in such a way that the liquid inside the liquid reservoir 31 is aspirated into the communicating passage 24 when a compressed gas is flown through the communicating passage 24, a constitution wherein the diameter of the communicating passage 24 is made larger than the liquid-spraying nozzle port 22 side via the step portion 24C while the liquid supply passage 25 is coupled in the vicinity of the step portion 24C within the large-diameter passage 24B of the communicating passage 24 having an expanded diameter, is sufficient.

In so doing, the angle at which the liquid supply passage 25 intersects the communicating passage 24 is not limited in particular, and an intersection that is approximately perpendicular, as in the present example, may be formed, or, additionally, an intersection that is inclined at any angle may also be formed.

The size of the diameter of the liquid supply passage 25 is preferably designed to be an appropriate size that is adequate to aspirate the liquid inside the liquid reservoir 31. In general, for instance, when the diameter of the large-diameter passage 24B is on the order of approximately 4 mm, the internal diameter of the liquid supply passage 25 is preferably on the order of approximately 1.5 mm. However, the size of the diameter is not limited thereto.

Method for Use

In the following, a method for mounting the liquid reservoir 31 onto the liquid-spraying adapter 20 for use will be described.

The interior of the liquid reservoir portion 32 of the liquid reservoir 31 is prefilled with a desired liquid A, and the opening of the coupling port 33 is openably covered with the blockage 40.

In so doing, to fill the liquid in the case where the blockage 40 is molded into one body with the liquid reservoir portion 32 and the like, it suffices, for instance, to open an injection pore at an appropriate location on the liquid reservoir portion 32, injecting the liquid therefrom and closing the pore thereafter.

In addition, in case a plastic sheet, film, or the like, which is a separate body, is used to cover, it suffices to cover the opening of the coupling port 33 with the plastic sheet, film, or the like after filling with the liquid.

Further additionally, in case of a constitution where a cap body 43 is attached, it suffices to attach the cap body 43 after filling with the liquid.

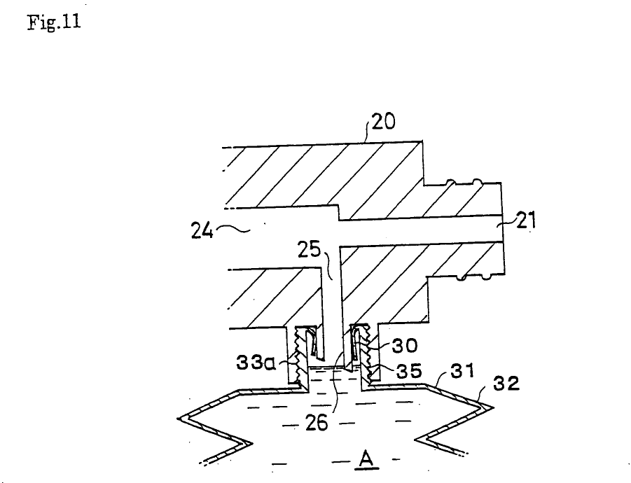

The liquid reservoir 31 that houses the liquid A in this way is coupled to the liquid reservoir coupling port 23 of the liquid-spraying adapter 20, as shown in FIG. 10 and FIG. 11. In so doing, packing is placed, as necessary, inside the coupling depression 23b of the liquid reservoir coupling port 23, and the circular tube portion 35 of the coupling port 33 is inserted into the coupling depression 23b and twisted in while screwing the screw portion 23c and the screw portion 33a. In this process, the tip of the tubular projection 26 pierces through the blockage 40 to open the blockage 40, and the tip of the tubular projection 26 penetrates into the liquid reservoir portion 32. By further twisting the circular tube portion 35 of the coupling port 33 into the coupling depression 23b of the liquid reservoir coupling port 23 up to a prescribed location, the coupling port 33 of the liquid reservoir 31 and the liquid reservoir coupling port 23 of the liquid-spraying adapter 20 can be coupled in a manner that maintains the airtight state.

By coupling the liquid reservoir 31 to the liquid-spraying adapter 20 in this way, since the coupling port 33 of the liquid reservoir 31 and the liquid reservoir coupling port 23 of the liquid-spraying adapter 20 can be joined in an intimate contact at the stage where the circular tube portion 35 of the coupling port 33 is inserted into the coupling depression 23b of the liquid reservoir coupling port 23, and the screw portion 23c and the screw portion 33a are starting to be screwed together, no leakage of the liquid occurs, even if the blockage 40 is opened subsequently.

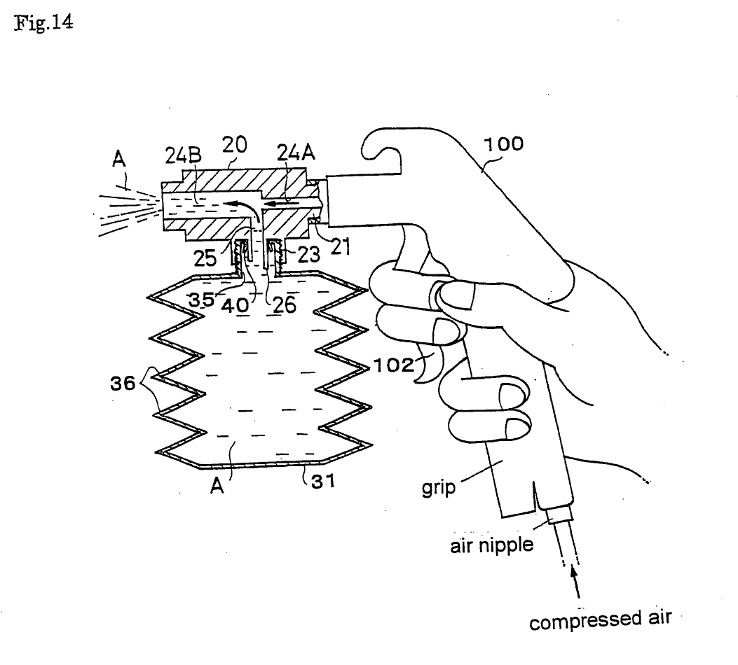

Then, when the nozzle 101 of the air gun 100 is connected to the connecting port 21 of the liquid-spraying adapter 20, as shown in FIG. 14, it is ready to spray.

Thereafter, as shown in FIG. 14, by operating the trigger 102 of the air gun 100 and spraying a compressed gas (compressed air) from the nozzle 101, the compressed gas can pass inside the communicating passage 24 and blow out from the liquid-spraying nozzle port 22, while at the same time, the compressed gas flowing through the communicating passage 24 aspirates the interior of the liquid reservoir 31. At that moment, since the interior of the liquid reservoir portion 32 of the liquid reservoir 31 is in a sealed state, when aspirating from the coupling port 33, the liquid reservoir portion 32 shrinks while the liquid A inside flows out from the coupling port 33 to the liquid supply passage 25 (it can also be stated that the liquid inside flows out from the coupling port 33 while the liquid reservoir portion 32 shrinks) and can be blown out together with the compressed gas (compressed air) via the communicating passage 24 (large-diameter passage 24B) from the liquid-spraying nozzle port 22.

Since the liquid reservoir 31 and the liquid-spraying adapter 20 function as mentioned above, a liquid can be continuously sprayed using a compressed gas supply apparatus or tool, such as an air gun, already installed in factories and other industrial institutions and the like. Moreover, since the interior of the liquid reservoir 31 is in a sealed state, the liquid reservoir 31 can be used without being upside-down, and the liquid can be sprayed equally by the air gun and the liquid-spraying adapter 20 at any angle spanning 360°.

Furthermore, it is possible to prevent the liquid inside the liquid reservoir 31 from leaking out, obviously when not coupled to the liquid-spraying adapter 20, but also when coupled to the liquid-spraying adapter 20.

Note that, it suffices to suitably form the coupling port 33 and the cap body 43 in relation to the coupling portion of the liquid-spraying adapter 20; for instance, if the liquid reservoir coupling port of the liquid-spraying adapter 20 is provided with a screw portion on the outer peripheral surface, the coupling port 33 must be provided with a screw portion on the inner peripheral surface that allows coupling thereto. To this end, for instance, it suffices to constitute a cap body 43 provided with a screw portion on the inner peripheral surface, so as to be mounted inside the circular tube portion 41.

In addition, the coupling structure between the coupling port of the liquid reservoir 31 and the coupling portion of the liquid-spraying adapter 20 is not limited to the screw-mount structure, as described above; for instance, fit-mount structure and other coupling structures can be adopted.

Although not shown, providing a hole on the bottom surface 32b of the projected portion 36A that is at the bottommost position among a plurality of projected portions 36 constituting the accordion portion 34 and constituting this hole to be openably closed, the liquid left inside the liquid reservoir portion can also be easily eliminated after use by opening this hole.

In addition, if the number of projected portions 36 constituting the accordion portion 34 is two steps or one step, internal volume when the accordion portion 34 is contracted can be made to be small, allowing the quantity of liquid left inside the liquid reservoir portion after use to be small.

In addition, it is also possible to attach a holder member that may maintain the contracted state of the accordion portion 34. For instance, a constitution allowing the most shrunk state of the accordion portion 34 to be maintained is possible by fixing one extremity of the holder member at the bottom surface 32b of the liquid reservoir portion 32 on one hand, forming a locking portion that locks the other extremity of the holder member at the top surface 32a of the liquid reservoir portion 32 or at the circular tube portion 35 on the other, and locking the other extremity of the holder member onto the lock portion with the accordion portion 34 in the most shrunk state.

In so doing, the holder member may have a string shape, a belt shape, a tape shape, or any other shape. The structure of the lock portion can also be formed in any way, such as a structure to wind the holder member, a structure to hook a hook portion, a structure to fasten with a button, a structure to fasten with a velvet fastener, and the like.

In addition, opposite to the above, one extremity of the holder portion may be fixed to the top surface of the liquid reservoir portion 32 or to the circular tube portion 35 and form the lock portion on the bottom surface 32b.

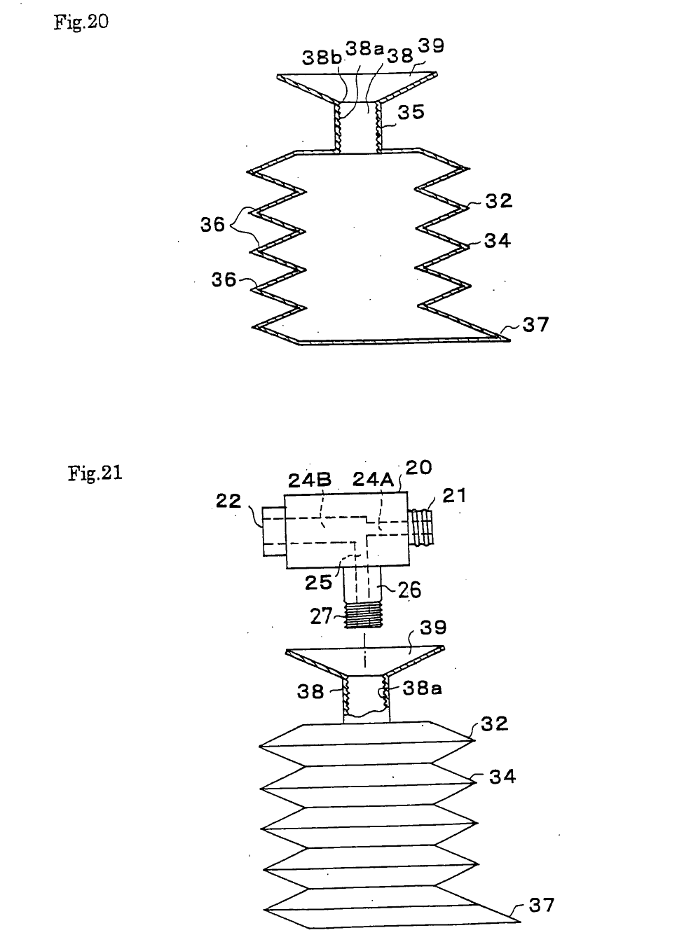

In addition, as shown in FIG. 20, a constitution is also possible wherein a screw portion 38a for coupling use is provided at the inner peripheral portion of the circular tube portion 35 of the coupling port to serve as a coupling port 38, and a liquid receiving portion 39 is provided, extending continuously and upward into a funnel shape from the upper edge portion 38b of this coupling port 38.

In this case, the liquid reservoir coupling port 23 of the liquid-spraying adapter 20 must be formed by providing a projection 26 on the bottom surface of the adapter main body 20A through which a liquid supply passage 25 passes and may be inserted into the circular tube portion 35, providing a screw portion 27 on the outer peripheral surface of this projection 26, to allow the liquid reservoir coupling port 23 and the coupling port 38 to be coupled, as shown in FIG. 21.

With this constitution, since the liquid receiving portion 39 just plays the role of a funnel, the liquid can be directly poured into the liquid reservoir portion 32 without spilling. Consequently, since when the liquid inside the liquid reservoir portion 32 becomes insufficient, the liquid reservoir 31 can be temporarily separated from the liquid-spraying adapter 20 and the liquid can be easily poured into the liquid reservoir portion 32, the liquid reservoir 31 can easily be repeatedly used.

Note that, such a constitution that provides a screw portion 38a for coupling use inside the coupling port 38 at the same time as providing a liquid receiving portion 39 can also be applied to a liquid reservoir with a constitution that does not provide the protrusion 37, in which case, the same effect as a liquid reservoir provided with the protrusion 37 can be obtained.

### Other Example of Liquid-Supplying Liquid Reservoir

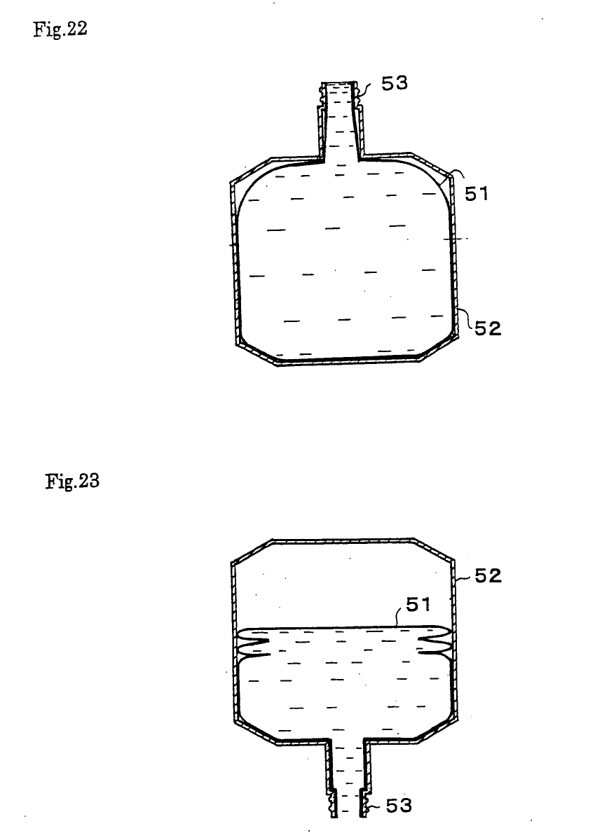

In addition, a liquid reservoir can be constituted as shown in FIG. 22 to FIG. 23.

That is to say, the liquid reservoir is not limited to the example described above, as long as it is provided with a liquid reservoir main body that can house a liquid and is expandable and contractible, and a coupling port. For instance, a liquid housing portion 51 that houses a liquid can also be formed with a plastic film bag, a rubber balloon bag, or the like, as shown in FIG. 22. In so doing, it is preferred that the liquid housing portion 51 is housed inside a form-retaining container 52, consisting of metal, plastic, or the like, in order to support the liquid housing portion 51. In such a constitution, since the liquid housing portion 51 is supported by the form-retaining container 52, it does not hinder work, even when it is upside-down, as shown in FIG. 23.

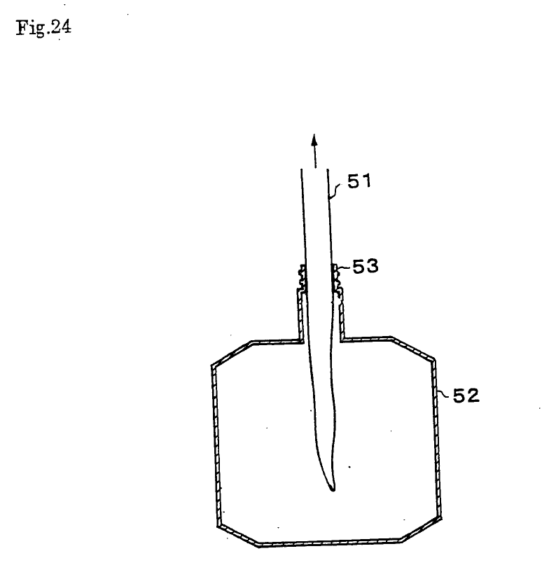

In addition, the coupling portion 53 is preferably provided on the form-retaining container 52, and the liquid housing portion 51 is preferably constituted removably with respect to the form-retaining container 52, as shown in FIG. 24.

### Example of Variation of Liquid-Spraying Adapter

In the liquid-spraying adapter of the present invention, if the connecting port has a constitution that allows coupling to the nozzle 101 of the air gun 100 in a manner that is removable and may retain airtightness, the connecting structure thereof can be formed at will. For instance, the connecting port can also be formed into a taper shape.

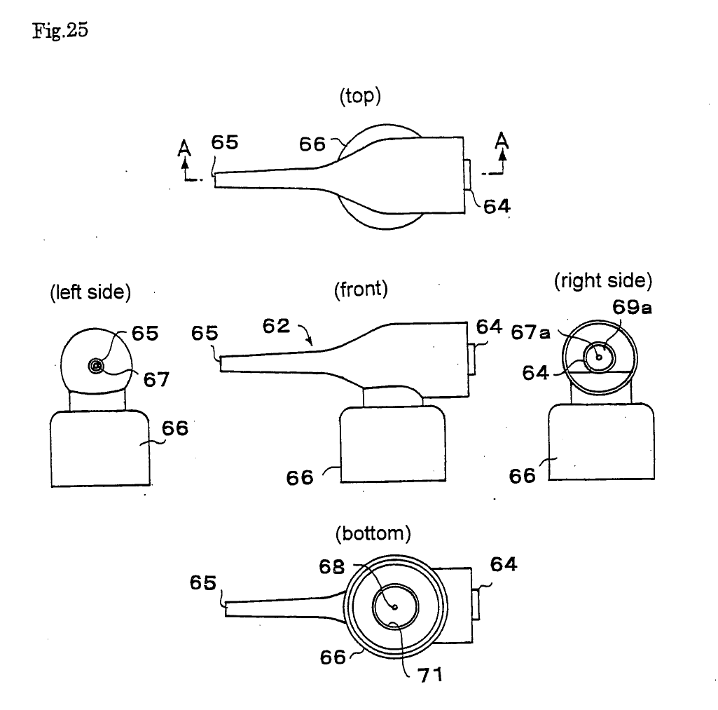

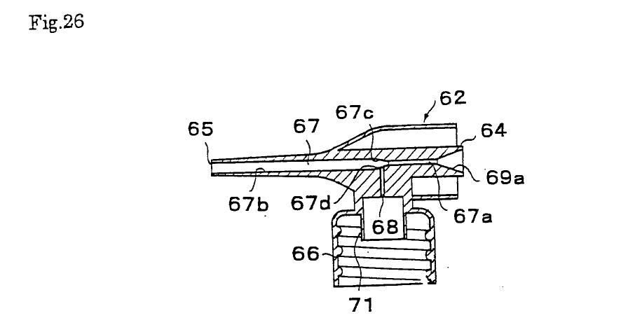

Specifically, it can be constituted as in FIG. 25 to 28. The liquid-spraying adapter 62 of this constitution example consists of an entire body of resin molded into one body with a funnel shape, as shown in FIG. 25 and FIG. 26, and, similarly to the adapter with the above-mentioned constitution, is constituted such that being provided with, respectively, a nozzle-receiving portion 64 having a taper surface 69a at one extremity thereof and a metal connecting port 65 at the other extremity; at the same time, a liquid reservoir coupling port 66 formed at the lower portion thereof, a nozzle-receiving portion 64 and the metal connecting port 65 communicating via a communicating passage 67; furthermore, the communicating passage 67 and a liquid reservoir coupling port 66 communicating via a liquid supply passage 68.

As shown in FIG. 26, the communicating passage 67 is such that a small-diameter passage 67a with a small opening diameter is on the nozzle-receiving portion 64 side, and a large-diameter passage 67b with a large opening diameter is on the metal connecting port 65 side, a step portion 67c where both passages connect to each other continuing the space from the small-diameter passage 67a end and the large-diameter passage 67b end with a taper surface 67d, and a liquid supply passage 68 running downward from the interior of this taper surface 67d.

In addition, so that it may fit on the outer periphery of the coupling portion 81 of the liquid reservoir 80 to assemble into one body, the liquid reservoir coupling port 66 has a shape wherein a circular cylindrical cap form, having a screw portion at the inner periphery, is given; at the center of the bottom surface of the circular cylindrical cap part thereof, a tubular tongue piece 71 with an appropriate length that joins to the inner periphery of the coupling portion 81 of the liquid reservoir 80 protrudes as one body, the open extremity of the liquid supply passage 68 being provided at the center of the tongue piece 71.

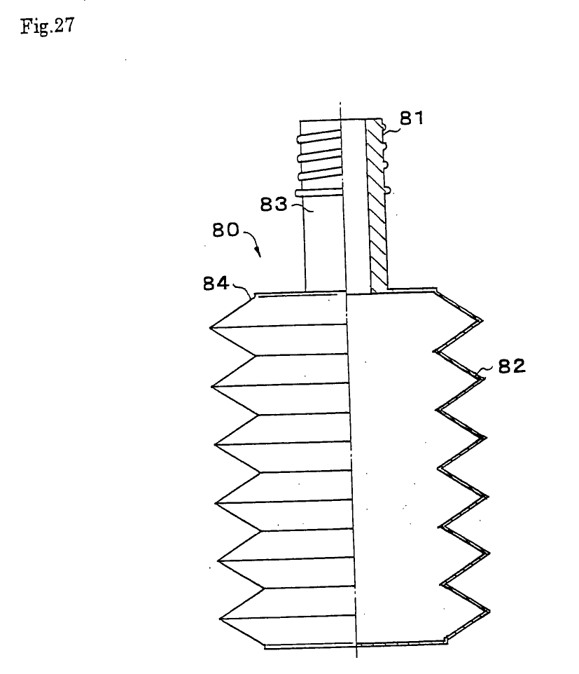

As shown in FIG. 27, the liquid reservoir 80 is constituted by a liquid reservoir main body 84 consisting of a retractable accordion-shaped body portion 82 that can hold a liquid with an appropriate capacity, from the top surface of which protrudes a circular tube portion 83. A coupling portion 81 on the outer peripheral surface, of which is engraved a thread groove, is provided on the upper portion of the circular tube portion 83, and the inside of this coupling portion 81 is provided as a cavity which the tongue piece 71 entirely fits in and joins with.

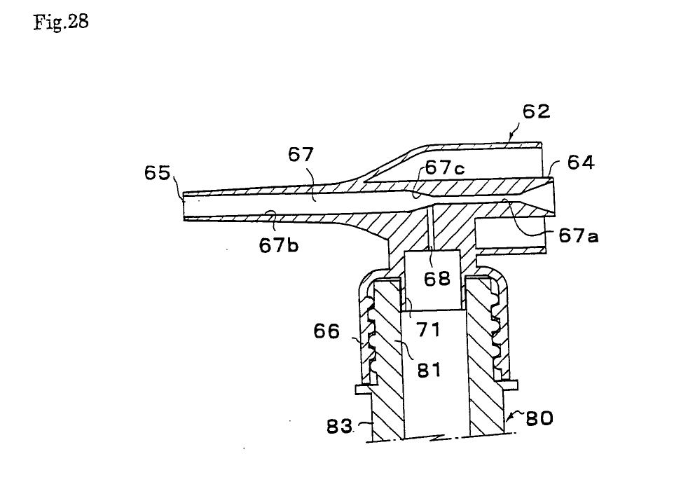

The liquid-spraying adapter constituted in this way is assembled by coupling the coupling portion 81 to the liquid reservoir coupling port 66 to have the liquid reservoir 80, in which the treatment solution has been placed, be mounted onto the liquid-spraying adapter 62. In this state, as shown in FIG. 28, the liquid reservoir coupling port 66 of the liquid-spraying adapter 62 fits onto the coupling portion 81 of the liquid reservoir 80 to close the opening of the liquid reservoir 80; furthermore, the tongue piece 71 inserts into the inner opening of the coupling portion 81 to join with the inner peripheral surface of the opening. That is to say, since the end opening of the circular tube portion 83 of the liquid reservoir 80 is closed by a double-wall surface of the liquid reservoir coupling port 66 and the tongue piece 71 with no spacing to be installed as one body at the lower portion of the liquid-spraying adapter 62, the air pressure inside the liquid reservoir 80 and the air pressure inside the liquid supply passage 68 are maintained constant, allowing the internal treatment solution to smoothly flow out via the liquid supply passage 68 into the communicating passage 67 and blow out to the exterior when a negative pressure occurs at the step portion 67c. In so doing, there is also no leakage of the treatment solution out from the junction with the liquid reservoir coupling port 66.

1. A liquid-spraying instrument, which is a liquid-spraying instrument that may be connected to a nozzle of a compressed gas supply means and provided with a liquid-spraying adapter and a liquid-supplying liquid reservoir, wherein
said liquid-spraying adapter is provided with a connecting port that may be removably connected to the nozzle of a compressed gas supply means, the liquid-spraying nozzle port that sprays a liquid, a liquid reservoir coupling port that may be removably coupled to a liquid-supplying liquid reservoir that houses a liquid, a communicating passage that communicates between said connecting port and said liquid-spraying nozzle port, and a liquid supply passage that communicates between said communicating passage and said liquid reservoir coupling port and may aspirate the liquid inside said liquid-supplying liquid reservoir when a compressed gas flows through said communicating passage.

2. The liquid-spraying instrument as recited in claim 1, wherein the nozzle of the compressed gas supply means is the nozzle of an air gun.

3. The liquid-spraying instrument as recited in claim 1 or 2 provided with a constitution, wherein the communicating passage of said liquid-spraying adapter is provided with a step portion at an intermediate portion, the connecting port side of said step portion being a small-diameter passage and the liquid-spraying nozzle port side of said step portion being a large-diameter passage whose diameter is larger than said small-diameter passage, and
said liquid supply passage establishing communication in the vicinity of said step portion within said large-diameter passage.

4. The liquid-spraying instrument as recited in any of claims 1 to 3, wherein said liquid-spraying adapter is provided with a selector switch that may open and close said liquid supply passage.

5. The liquid-spraying instrument as recited in any of claims 1 to 4, wherein a spray adjustment tool that may adjust the spraying amount, spray shape, and the like is connected to said liquid-spraying nozzle port of said liquid-spraying adapter.

6. The liquid-spraying instrument as recited in any of claims 1 to 5, wherein said liquid reservoir coupling port of said liquid-spraying adapter is provided with a pointed tubular projection having a liquid supply passage on the inside.

7. The liquid-spraying instrument as recited in claim 1 with a constitution wherein said liquid-supplying liquid reservoir is provided with a liquid reservoir portion that is capable of housing a liquid and is expandable and contractible, and a coupling port/liquid supply port, the liquid inside being sent out from the coupling port/liquid supply port while the liquid reservoir portion contracts, when the interior of the liquid reservoir portion is aspirated via said coupling port/liquid supply port.

8. The liquid-spraying instrument as recited in claim 7, wherein said liquid-supplying liquid reservoir is one with the opening of said coupling port/liquid supply port openably closed.

9. The liquid-spraying instrument as recited in claim 8, wherein the opening of said coupling port/liquid supply port of said liquid-supplying liquid reservoir is closed with a plastic film or sheet.

10. The liquid-spraying instrument as recited in claim 7 with a constitution, wherein said coupling port/liquid supply port of said liquid-supplying liquid reservoir is constituted in such a way that it may have a cap body attached, whose top surface is formed to be openable, the opening of said coupling port/liquid supply port being openably closed by attaching said cap body.

11. A liquid-spraying adapter, which is a liquid-spraying adapter that connects to the nozzle of a compressed gas supply means, provided with
a connecting port that may be removably connected to the nozzle of the compressed gas supply means, a liquid-spraying nozzle port that sprays a liquid, a liquid reservoir coupling port that may be removably coupled to a liquid-supplying liquid reservoir that houses a liquid, a communicating passage that communicates between said connecting port and said liquid-spraying nozzle port, and a liquid supply passage that communicates between said communicating passage and said liquid reservoir coupling port and may aspirate the liquid inside said liquid-supplying liquid reservoir when a compressed gas flows through said communicating passage.

12. A liquid-supplying liquid reservoir provided with a constitution, wherein a liquid reservoir portion that is capable of housing a liquid and is expandable and contractible, and a coupling port/liquid supply port are provided, the liquid inside being sent out from the coupling port/liquid supply port while the liquid reservoir portion contracts, when the interior of the liquid reservoir portion is aspirated via said coupling port/liquid supply port.
